(19) United States
(12) Patent Application Publication
(10) Pub. No.: US 2025/0164469 A1
(43) Pub. Date: May 22, 2025

(54) HIGH-SENSITIVITY HAPTEN DETECTION METHOD AND DEVICE

(71) Applicant: National Yang Ming Chiao Tung University, Taipei City (TW)

(72) Inventors: How-Foo CHEN, Taipei City (TW); Yu-Chun LIN, Taipei City (TW); Chih-Han CHEN, Taipei City (TW)

(21) Appl. No.: 18/420,198

(22) Filed: Jan. 23, 2024

(30) Foreign Application Priority Data
Nov. 22, 2023 (TW) 112145062

(51) Int. Cl. G01N 33/53; G01N 33/553

(52) U.S. Cl. CPC G01N 33/5308; G01N 33/553

(57) ABSTRACT

A high-sensitivity hapten detection method capable of deriving a real time hapten concentration by using a SPR device, in which different concentrations of a hapten in a sample solution result in different reflected light intensities on the SPR device, and by mapping a reflected light intensity, via a lookup table, to a corresponding concentration, a detection result of the concentration of the hapten in the sample solution can be instantly provided.

### BACKGROUND OF THE INVENTION

### Field of the Invention

The invention relates to hapten detection methods, especially to an optical hapten detection method.

### Description of the Related Art

General contaminants of water or food include haptenic substance, such as pesticides, antibiotics, malachite green, preservatives, pesticides, or melamine.

In addition, the common method for detecting haptenic substance is enzyme-linked immunosorbent assay (ELISA). However, the disadvantage of this method is that it is not possible to get the detection result on-site in real time.

To solve the above mentioned problem, there is an urgent need for a novel hapten detection method in the field.

### SUMMARY OF THE INVENTION

The main objective of the invention is to provide a high-sensitivity hapten detection method, which can derive a real time hapten concentration by using a SPR (surface plasmon resonance) device to detect a reflected light intensity caused by a sample solution containing a hapten. As different concentrations of the hapten result in different reflected light intensities on the SPR device, by mapping a reflected light intensity, via a lookup table, to a corresponding concentration, the invention can instantly provide a detection result of the concentration of the hapten.

To achieve the above objective, a high-sensitivity hapten detection method is proposed for detecting a concentration of a hapten in a sample solution, including:
adding a first volume of a buffer solution to each of a first sample area and a second sample area, and then performing a reflected light intensity detection operation on each of the first sample area and the second sample area to obtain a first reflected light intensity and a second reflected light intensity respectively;
adding a second volume of the sample solution and an amount of an antibody to the first sample area, adding the second volume of the sample solution to the second sample area, and after a period of time adding a third volume of the buffer solution to each of the first sample area and the second sample area, and then performing the reflected light intensity detection operation on each of the first sample area and the second sample area to obtain a third reflected light intensity and a fourth reflected light intensity respectively; and
generating a first difference value according to a difference between the first reflected light intensity and the third reflected light intensity, generating a second difference value according to a difference between the second reflected light intensity and the fourth reflected light intensity, and generating a third difference value according to a difference between the first difference value and the second difference value, where the third difference value is positively correlated with the concentration of the hapten.

In one embodiment, the reflected light intensity detection operation includes: using a light source to emit a light beam with an incident angle to illuminate a reflective surface of a test zone, the test zone being in contact with a local area of the first sample area and a local area of the second sample area; and using a photo sensor to detect the intensity of the reflected light of the test zone.

In one embodiment, the reflective surface of the test zone is covered with a layer of gold film, and the layer of gold film is coated with the hapten.

In one embodiment, the concentration of the hapten is obtained by using a processor to map the third difference value, via a lookup table, to an output value, and the lookup table is stored in a memory.

In one embodiment, the hapten is malachite green and the antibody is bonded with nanoparticles.

To attain the above objective, the invention further proposes a high-sensitivity hapten detection device, which includes a reflected light intensity detection device, a first sample area, a second sample area, and a processor to perform a hapten detection procedure to detect a concentration of a hapten in a sample solution, the hapten detection procedure including:
adding a first volume of a buffer solution to each of a first sample area and a second sample area, and then using the reflected light intensity detection device to perform a reflected light intensity detection operation on each of the first sample area and the second sample area to obtain a first reflected light intensity and a second reflected light intensity respectively;
adding a second volume of the sample solution and an amount of an antibody to the first sample area, adding the second volume of the sample solution to the second sample area, and after a period of time adding a third volume of the buffer solution to each of the first sample area and the second sample area, and then using the reflected light intensity detection device to perform the reflected light intensity detection operation on each of the first sample area and the second sample area to obtain a third reflected light intensity and a fourth reflected light intensity respectively; and
the processor generating a first difference value according to a difference between the first reflected light intensity and the third reflected light intensity, generating a second difference value according to a difference between the second reflected light intensity and the fourth reflected light intensity, and generating a third difference value according to a difference between the first difference value and the second difference value, where the third difference value is positively correlated with the concentration of the hapten.

In one embodiment, the reflected light intensity detection operation includes: using a light source of the reflected light intensity detection device to emit a light beam with an incident angle to illuminate a reflective surface of a test zone, the test zone being in contact with a local area of the first sample area and a local area of the second sample area; and using a photo sensor of the reflected light intensity detection device to detect the intensity of the reflected light of the test zone.

In one embodiment, the reflective surface of the test zone is covered with a layer of gold film, and the layer of gold film is coated with the hapten.

In one embodiment, the concentration of the hapten is obtained by using the processor to map the third difference value, via a lookup table, to an output value, and the lookup table is stored in a memory.

In one embodiment, the hapten is malachite green and the antibody is bonded with nanoparticles.

To make it easier for our examiner to understand the objective of the invention, its structure, innovative features, and performance, we use preferred embodiments together with the accompanying drawings for the detailed description of the invention.

### BRIEF DESCRIPTION OF THE DRAWINGS

FIG. 1 illustrates an embodiment of the hapten detection device of the invention;

FIG. 2 illustrates two distribution curves over time of two groups of detection values obtained by the processor of the hapten detection device of FIG. 1, the two groups of detection values being derived from the reflected light intensities of the first sample area and the second sample area; and

FIG. 3 illustrates a flowchart of a high-sensitivity hapten detection method according to one embodiment of the present invention.

### DETAILED DESCRIPTION OF THE PREFERRED EMBODIMENTS

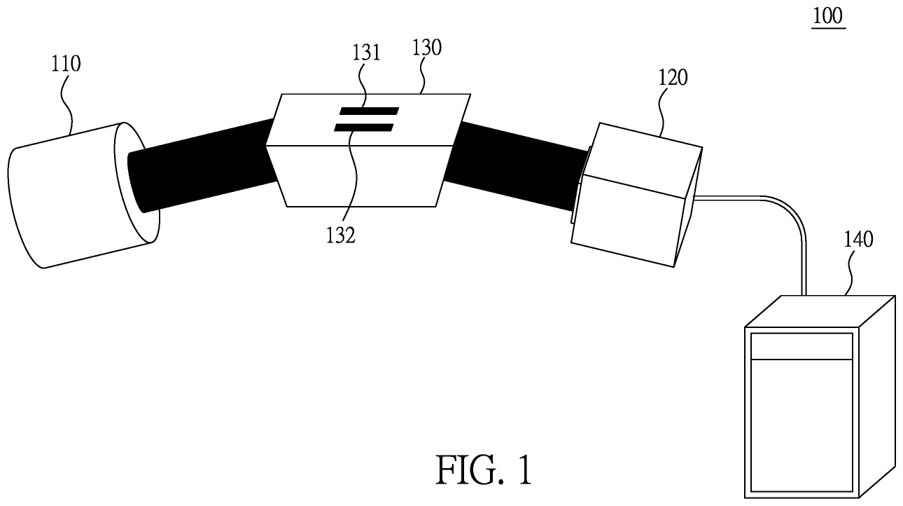

Please refer to FIG. 1, which illustrates an embodiment of the hapten detection device of the invention. As shown in FIG. 1, a hapten detection device 100 includes a light source 110, a reflected light intensity detection device 120, a detection platform 130, and a processor 140 to perform a hapten detection procedure for detecting a concentration of a hapten in a sample solution, where the detection platform 130 has a first sample area 131 and a second sample area 132.

To be specific, the hapten detection procedure includes:

(A) Add a first volume of a buffer solution to each of the first sample area 131 and the second sample area 132, and then use the reflected light intensity detection device 120 to perform a reflected light intensity detection operation on each of the first sample area 131 and the second sample area 132 to obtain a first reflected light intensity and a second reflected light intensity respectively, where the buffer solution can be prepared from a mixture containing a weak acid and the conjugate base of the weak acid or a weak base and the conjugate acid of the weak base.

(B) Add a second volume of the sample solution and an amount of an antibody to the first sample area 131, add the second volume of the sample solution to the second sample area 132, and after a period of time add a third volume of the buffer solution to each of the first sample area 131 and the second sample area 132, and then use the reflected light intensity detection device 120 to perform the reflected light intensity detection operation on each of the first sample area 131 and the second sample area 132 to obtain a third reflected light intensity and a fourth reflected light intensity respectively.

(C) The processor 140 generates a first difference value according to a difference between the first reflected light intensity and the third reflected light intensity, generates a second difference value according to a difference between the second reflected light intensity and the fourth reflected light intensity, and generates a third difference value according to a difference between the first difference value and the second difference value, where the third difference value is positively correlated with the concentration of the hapten. In addition, the processor 140 can further divide the third difference value with a detection sensitivity coefficient to generate a normalized detection value to represent the concentration, or map the third difference value, via a lookup table, to an output value to represent the concentration, where the lookup table is stored in a memory.

To be specific, the reflected light intensity detection operation includes: using the light source 110 to emit a light beam with an incident angle to illuminate a reflective surface (not shown in the figure) of a test zone, the test zone being in contact with a local area of the first sample area 131 and a local area of the second sample area 132; and using a photo sensor of the reflected light intensity detection device 120 to detect the intensity of the reflected light of the test zone; where the reflective surface of the test zone is covered with a layer of gold film, and the layer of gold film can be coated with the hapten to optimize the detection effect.

In addition, the antibody can be bonded with nanoparticles, and the hapten can be a small-molecule substance such as pesticide, antibiotic, malachite green, preservative, pesticide, or melamine.

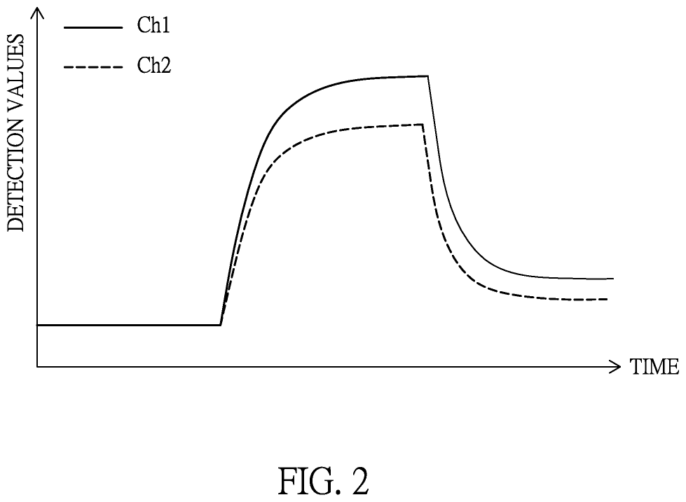

In addition, the invention utilizes the hapten detection device 100 of FIG. 1 to perform a detection operation on malachite green. Please refer to FIG. 2, which illustrates two distribution curves over time of two groups of detection values obtained by the processor 140 of the hapten detection device 100 of FIG. 1, the two groups of detection values being derived from the reflected light intensities of the first sample area 131 and the second sample area 132. As shown in FIG. 2, the detection values obtained from the first sample area 131 is significantly higher than the detection values obtained from the second sample area 132.

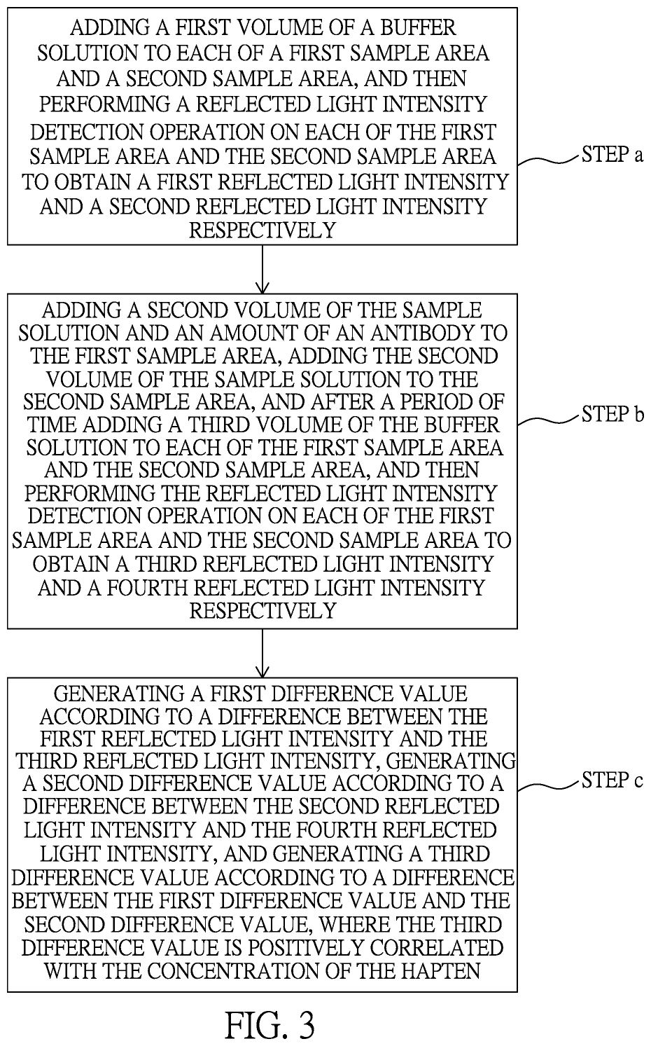

As can be seen from the description above, the invention proposes a high-sensitivity hapten detection method. Please refer to FIG. 3, which illustrates a flowchart of a high-sensitivity hapten detection method according to one embodiment of the present invention for detecting a concentration of a hapten in a sample solution. As shown in FIG. 3, the method includes: adding a first volume of a buffer solution to each of a first sample area and a second sample area, and then performing a reflected light intensity detection operation on each of the first sample area and the second sample area to obtain a first reflected light intensity and a second reflected light intensity respectively (step a); adding a second volume of the sample solution and an amount of an antibody to the first sample area, adding the second volume of the sample solution to the second sample area, and after a period of time adding a third volume of the buffer solution to each of the first sample area and the second sample area, and then performing the reflected light intensity detection operation on each of the first sample area and the second sample area to obtain a third reflected light intensity and a fourth reflected light intensity respectively (step b); and generating a first difference value according to a difference between the first reflected light intensity and the third reflected light intensity, generating a second difference value according to a difference between the second reflected light intensity and the fourth reflected light intensity, and generating a third difference value according to a difference between the first difference value and the second difference value, where the third difference value is positively correlated with the concentration of the hapten (step c).

In the steps mentioned above, the reflected light intensity detection operation includes: using a light source to emit a light beam with an incident angle to illuminate a reflective surface of a test zone, the test zone being in contact with a local area of the first sample area and a local area of the second sample area; and using a photo sensor to detect the intensity of the reflected light of the test zone.

In addition, the reflective surface of the test zone is covered with a layer of gold film, and the layer of gold film can be coated with the hapten to optimize the detection effect.

In addition, the method can utilize a processor to divide the third difference value with a detection sensitivity coefficient to generate a normalized detection value to represent the concentration, or map the third difference value, via a lookup table, to an output value to represent the concentration, where the lookup table is stored in a memory.

In addition, the antibody can be bonded with nanoparticles, and the hapten can be a small-molecule substance such as pesticide, antibiotic, malachite green, preservative, pesticide, or melamine.

With the designs disclosed above, the invention offers the advantages as follows:

The invention can derive a real time hapten concentration by using a SPR (surface plasmon resonance) device to detect a reflected light intensity caused by a sample solution containing a hapten. As different concentrations of the hapten result in different reflected light intensities on the SPR device, by mapping a reflected light intensity, via a lookup table, to a corresponding concentration, the invention can instantly provide a detection result of the concentration of the hapten.

While the invention has been described by way of example and in terms of preferred embodiments, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

In summation of the above description, the present invention herein enhances the performance over the conventional structure and further complies with the patent application requirements and is submitted to the Patent and Trademark Office for review and granting of the commensurate patent rights.

What is claimed is:

1. A high-sensitivity hapten detection method for detecting a concentration of a hapten in a sample solution, including:
adding a first volume of a buffer solution to each of a first sample area and a second sample area, and then performing a reflected light intensity detection operation on each of the first sample area and the second sample area to obtain a first reflected light intensity and a second reflected light intensity respectively;
adding a second volume of the sample solution and an amount of an antibody to the first sample area, adding the second volume of the sample solution to the second sample area, and after a period of time adding a third volume of the buffer solution to each of the first sample area and the second sample area, and then performing the reflected light intensity detection operation on each of the first sample area and the second sample area to obtain a third reflected light intensity and a fourth reflected light intensity respectively; and
generating a first difference value according to a difference between the first reflected light intensity and the third reflected light intensity, generating a second difference value according to a difference between the second reflected light intensity and the fourth reflected light intensity, and generating a third difference value according to a difference between the first difference value and the second difference value, wherein the third difference value is positively correlated with the concentration of the hapten.

2. The high-sensitivity hapten detection method as disclosed in claim 1, wherein the reflected light intensity detection operation includes: using a light source to emit a light beam with an incident angle to illuminate a reflective surface of a test zone, the test zone being in contact with a local area of the first sample area and a local area of the second sample area; and using a photo sensor to detect a reflected light intensity of the test zone.

3. The high-sensitivity hapten detection method as disclosed in claim 2, wherein the reflective surface of the test zone is covered with a layer of gold film, and the layer of gold film is coated with the hapten.

4. The high-sensitivity hapten detection method as disclosed in claim 1, wherein the concentration of the hapten is obtained by using a processor to map the third difference value, via a lookup table, to an output value, and the lookup table is stored in a memory.

5. The high-sensitivity hapten detection method as disclosed in claim 1, wherein the hapten is malachite green and the antibody is bonded with nanoparticles.

6. A high-sensitivity hapten detection device, which includes a reflected light intensity detection device, a first sample area, a second sample area, and a processor to perform a hapten detection procedure to detect a concentration of a hapten in a sample solution, the hapten detection procedure including:
adding a first volume of a buffer solution to each of a first sample area and a second sample area, and then using the reflected light intensity detection device to perform a reflected light intensity detection operation on each of the first sample area and the second sample area to obtain a first reflected light intensity and a second reflected light intensity respectively;
adding a second volume of the sample solution and an amount of an antibody to the first sample area, adding the second volume of the sample solution to the second sample area, and after a period of time adding a third volume of the buffer solution to each of the first sample area and the second sample area, and then using the reflected light intensity detection device to perform the reflected light intensity detection operation on each of the first sample area and the second sample area to obtain a third reflected light intensity and a fourth reflected light intensity respectively; and
the processor generating a first difference value according to a difference between the first reflected light intensity and the third reflected light intensity, generating a second difference value according to a difference between the second reflected light intensity and the fourth reflected light intensity, and generating a third difference value according to a difference between the first difference value and the second difference value, wherein the third difference value is positively correlated with the concentration of the hapten.

7. The high-sensitivity hapten detection device as disclosed in claim 6, wherein the reflected light intensity detection operation includes: using a light source of the reflected light intensity detection device to emit a light beam with an incident angle to illuminate a reflective surface of a test zone, the test zone being in contact with a local area of the first sample area and a local area of the second sample area; and using a photo sensor of the reflected light intensity detection device to detect a reflected light intensity of the test zone.

8. The high-sensitivity hapten detection device as disclosed in claim 7, wherein the reflective surface of the test zone is covered with a layer of gold film, and the layer of gold film is coated with the hapten.

9. The high-sensitivity hapten detection device as disclosed in claim 6, wherein the concentration of the hapten is obtained by using the processor to map the third difference value, via a lookup table, to an output value, and the lookup table is stored in a memory.

10. The high-sensitivity hapten detection device as disclosed in claim 7, wherein the hapten is malachite green and the antibody is bonded with nanoparticles.
